(19) United States
(12) Patent Application Publication
(10) Pub. No.: US 2023/0092495 A1
(43) Pub. Date: Mar. 23, 2023

(54) SOLID FORMS OF TEZACAFTOR AND PROCESSES FOR THE PREPARATION THEREOF

(71) Applicant: QUÍMICA SINTÉTICA, S.A., ALCALÁ DE HENARES (ES)

(72) Inventors: Giuseppe BARRECA, Sirtori (IT); Luca CARCONE, Milano (IT); Marianna COPPOLA, Vercelli (IT); Cristina PESENTI, Gorla Minore (IT); Massimo ZAMPIERI, Cesano Maderno (IT); Davide BORDONE, Vercelli (IT); Silvano RONZONI, Milano (IT); Piero PARAVIDINO, Sedriano (IT)

(21) Appl. No.: 17/909,351

(22) PCT Filed: Mar. 5, 2021
(86) PCT No.: PCT/EP2021/055612

§ 371 Date: Sep. 2, 2022

(30) Foreign Application Priority Data
Mar. 6, 2020 (EP) 20382165.7
Sep. 15, 2020 (EP) 20382811.6

(51) Int. Cl. C07D 405/12

(52) U.S. Cl. CPC C07D 405/12; C07B 2200/13

(57) ABSTRACT

A crystalline form of tezacaftor, an industrially viable and advantageous process for its preparation and a method for the preparation of amorphous form of tezacaftor are described.

This application claims the benefit of European Patent Application EP20382165.7 filed on 6 Mar. 2020 and of the European Patent Application EP20382811.6 filed on 15 Sep. 2020.

### FIELD OF THE INVENTION

The present invention relates to solid forms of tezacaftor suitable for use in therapy, e.g. in the treatment of CFTR-mediated diseases, such as cystic fibrosis, and to an industrially viable and advantageous process for the preparation of said solid forms of tezacaftor.

### BACKGROUND ART

CFTR is a cAMP/ATP-mediated anion channel that is expressed in a variety of cells types, including absorptive and secretory epithelia cells, where it regulates anion flux across the membrane, as well as the activity of other ion channels and proteins. In epithelia cells, normal functioning of CFTR is critical for the maintenance of electrolyte transport throughout the body, including respiratory and digestive tissue. CFTR is composed of approximately 1480 amino acids that encode a protein made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking. A defect in the gene encoding CFTR causes mutations resulting in cystic fibrosis (“CF”), the most common fatal genetic disease in humans.

Tezacaftor is the INN denomination assigned to the compound having chemical name (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide. Its chemical structure is:

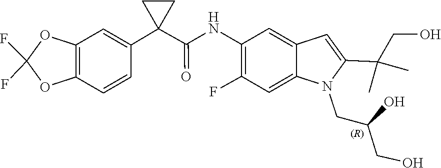

Tezacaftor and its use in the treatment of cystic fibrosis were first disclosed in the US patent application US 2009/0131492. The process described therein entails isolation of the final product as a cream coloured foamy solid by column chromatography using a mixture of ethyl acetate-hexane.

As generally known, any active principle may exist under amorphous or different crystalline forms (polymorphs), either as pure compound or in forms in which in the structure of the crystal are present molecules of water (hydrates) or of another solvent (solvates); besides, in case of hydrates and solvates, the ratio between the number of molecules of active principle and molecules of water or solvent may vary, giving rise to different solid forms of the compound.

In particular, amorphous solids consist of disordered arrangement of molecules and do not possess a distinguishable crystal lattice.

A new solid form of a compound may possess physical properties that differ from, and are advantageous over, those of other crystalline modifications. These include packing properties such as molar volume and density; thermodynamic properties such as glass transition temperature and solubility; kinetic properties such as dissolution rate; surface properties such as wettability interfacial tension; handling and filtration properties and so on. Variations in any of these properties may affect the chemical and pharmaceutical processing of a compound and may often render a new form more suitable for pharmaceutical and medical use.

WO 2011/119984 A1 discloses two polymorphs of Tezacaftor, referred to as Form A and amorphous form. Form A is characterized by a powder X-ray diffraction pattern (XRPD) having the main peaks at about 10.0°, 17.1°, 20.75°, 18.8°, 19.5°, 20.4°, 21.7° and 24.7° 2θ while the amorphous form is characterized by an XRPD profile having a halo pattern between 12° and 32° 2θ.

According to WO 2011/119984 said amorphous form of tezacaftor can be prepared by evaporating, at 50° C., a solution of tezacaftor in methanol.

On industrial scale, the preparation of an amorphous form through distillation of the solvent under normal or reduced pressure, as described in US 2009/0131492 and in WO 2011/119984 A1, is of difficult implementation at least to the extent that it is lengthy, difficult to control and requires the use of specialized equipment not always available in a multi-purpose plant. Also the methods for the preparation of form A disclosed in WO 2011/119984 A1, such as slurring, fast or slow evaporation of a solvent and addition of an anti-solvent to a solution obtained after sonicating, appear unsuitable for scaling-up and production on industrial scale.

An object of this invention is, therefore, to provide a new stable crystalline form of tezacaftor and a new method for its preparation in yields and purity adequate for pharmaceutical use that is feasible, reliable and suitable for scaling up.

Another object of this invention is to provide new methods for the preparation of a stable amorphous form of tezacaftor with yields and in a purity adequate for pharmaceutical use.

### SUMMARY OF THE INVENTION

These objectives are achieved with the present invention that, in an aspect thereof, relates to a polymorphically and/or chemically stable crystalline form of (R)-1-(2,2-difluorobenzo[d][1,3]dioxol-5-yl)-N-(1-(2,3-dihydroxypropyl)-6-fluoro-2-(1-hydroxy methylpropan-2-yl)-1H-indol-5-yl)cyclopropane-1-carboxamide (tezacaftor), preferably a polymorphically and chemically stable crystalline form of tezacaftor (hereafter also referred to as crystalline form B), said stable crystalline form being characterized by an XRPD profile comprising at least the peaks at 5.4-5.8, 11.1-11.5, 12.7- 13.1 and 15.7-16.1 degrees 2θ, when collected with the Kα radiation of copper (λ=1.5418 Å).

In another aspect, the present invention relates to a process for the preparation of said stable crystalline form B of tezacaftor.

In another aspect, the invention relates to a pharmaceutical composition comprising said stable crystalline form B of tezacaftor in mixture with one or more pharmaceutically acceptable excipient(s) and to a method for preparing said pharmaceutical composition.

In another aspect, the invention relates to the use of the stable crystalline form B of tezacaftor as described above for preparing other solid-state forms of tezacaftor.

The present invention, in another aspect, relates to a stable amorphous form of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor), which shows an XRPD profile comprising a halo pattern between 8° and 33° 2θ, when collected with the Kα radiation of copper (λ=1.5418 Å).

In another aspect, the invention relates to several processes for producing said stable amorphous form of tezacaftor.

In yet another aspect, the invention relates to a pharmaceutical composition comprising the stable amorphous form of tezacaftor in mixture with one or more pharmaceutically acceptable excipient.

In another aspect, the invention relates to the use of the stable amorphous form of tezacaftor for preparing other solid-state forms of tezacaftor.

### BRIEF DESCRIPTION OF THE DRAWINGS

FIG. 1 depicts the X-Ray powder diffractogram of the stable crystalline form of the invention, hereafter also indicated as form B.

FIG. 2 shows a comparison between the X-Ray powder diffractogram of the stable crystalline form of the invention, hereafter also indicated as form B, and the corresponding diffractogram of form A (prepared according to WO2011/119984).

FIG. 3 shows a DSC thermogram of tezacaftor crystal Form B according to the invention.

FIG. 4 depicts the X-Ray powder diffractogram of the stable amorphous form of the invention.

### DETAILED DESCRIPTION OF THE INVENTION

All terms used in this application, unless otherwise specified, are to be understood in their ordinary meaning as known in the technical field.

The term “about” includes the range of experimental errors, which can normally occur performing a measurement, e.g. ±5% or ±2% or ±1%.

The term “mass” defines the combination of substrates, reagents, solvents, and products on which a physical or chemical transformation is carried out.

The term “excipient” means any substance contained in the final pharmaceutical form other than the active ingredient and which generally may not be therapeutically effective by itself.

Excipients are essential for the administration of the active substance, as they allow to deliver the drug to the target site. Excipients are commonly referred to as raw materials entering into the composition of a pharmaceutical preparation with the aim of giving a shape, to facilitate administration and preserve the active ingredient. Furthermore, they contribute to characterize the pharmaceutical preparation from the point of view of appearance, stability, biopharmaceutical profile and acceptability by the patient.

Unless otherwise indicated, in the context of the present invention the percentage and amount of a certain component in a composition are to be referred to the weight of said component with respect to the total weight of the composition.

Unless otherwise indicated, in the context of the present invention the indication that a composition “comprises”other one or more components/elements means that the indicated components/elements must be present and also other components may be present, but are not necessarily present, in the composition, in addition to the ones specifically recited. In other words, the indication that a composition “comprises” one or more components does not exclude that the composition consists of, or consists essentially of, the recited component(s).

As used herein, the indication that a compound or composition A is “entirely free” of other substances (or “consists of”) means that, within the detection range of the instrument or method being used, no substances other than those specifically indicated can be detected in A.

As used herein, the term “a compound or composition A is essentially free of other substance(s)”, or “consists essentially of A”, means that only trace amount of substance(s) other than A, if any, can be detected using the analytical methods and techniques known to the person skilled in the art.

Unless otherwise indicated, in the context of the present invention a range of values indicated for a certain parameter, for example the weight of a component in a mixture, includes the upper and the lower limits of the range, e.g. if the content in weight, or in volume, of a component A in a mixture is indicated as “X to Y”, the content of A can be X, Y or any of the intermediate values. By “polymorphically stable” it is meant that the crystalline form B and the amorphous form of the present invention, when stored (I) at 70° C. under reduced pressure for at least 1 hour (preferably for 5 hours, more preferably for 10 hours, even more preferably for 12 hours), (II) at 60° C. for at least 1 day (preferably for 5 days, more preferably for 10 days, even more preferably for 15 days), (III) at 40° C. and 75% RH for at least 1 day (preferably for 5 days, more preferably for 10 days, even more preferably for 15 days), and/or (IV) at 25-30° C. and 80% RH for at least 1 day (preferably for 5 days, more preferably for 10 days, even more preferably for 15 days), show no signs of transformation into a different crystalline tezacaftor (e.g. Form A) as evaluated by the absence of peaks in an X-ray powder diffractogram (XRPD).

By “chemically stable” it is meant that the crystalline form B and the amorphous form of the present invention show no degradation upon storage under stressed conditions, e.g. when stored (I) at 70° C. under reduced pressure for at least 1 hour (preferably for 5 hours, more preferably for 10 hours, even more preferably for 12 hours), (II) at 60° C. for at least 1 day (preferably for 5 days, more preferably for 10 days, even more preferably for 15 days), (III) at 40° C. and 75% RH for at least 1 day (preferably for 5 days, more preferably for 10 days, even more preferably for 15 days), and/or (IV) at 25-30° C. and 80% RH for at least 1 day (preferably for 5 days, more preferably for 10 days, even more preferably for 15 days). “No degradation” means that a HPLC analysis of tezacaftor shows no significant worsening of the purity, in terms of formation of new impurities and increase of the content of those already present profile with respect to the initial profile (for example, less than 0.1% area increase).

Unless otherwise indicated, the data relative to the peaks in the XRPD pattern are meant within the common uncertainty due to the instrument measurement, typically ±0.2 degrees 2θ, when collected with the Kα radiation of copper (λ=1.5418 Å).

In one aspect, the present invention relates to a polymorphically and/or chemically stable crystalline form of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor), preferably a polymorphically and chemically stable crystalline form of tezacaftor, said stable crystalline form being characterized by an X-ray powder diffraction (XRPD) profile comprising at least the peaks at 5.4-5.8, 11.1-11.5, 12.7-13.1 and 15.7-16.1 degrees 2θ, when collected with the Kα radiation of copper (λ=1.5418 Å).

Said stable crystalline form having an XRPD profile comprising at least each of the peaks at 5.4-5.8, 11.1-11.5, 12.7-13.1 and 15.7-16.1 degrees 2θ (and, additionally, other peaks) when collected with the Kα radiation of copper (λ=1.5418 Å) is hereafter also referred to as crystalline form B of tezacaftor.

More preferably, said stable crystalline form B of tezacaftor is characterized by an XRPD profile comprising at least peaks at 5.6-5.7, 11.3-11.4, 12.80-12.9 and 15.80-16.0 degrees 2θ, when collected with the Kα radiation of copper (λ=1.5418 Å).

More preferably, said stable crystalline form B of tezacaftor is characterized by an XRPD profile comprising at least peaks at 5.6, 11.3, 12.9 and 15.9 degrees 2θ, when collected with the Kα radiation of copper (λ=1.5418 Å).

More preferably, said stable crystalline form B of tezacaftor is characterized by an XRPD profile additionally comprising at least one of the peaks at 16.3, 19.6, 20.6, 21.3, 26.9 and 31.4 degrees 2θ.

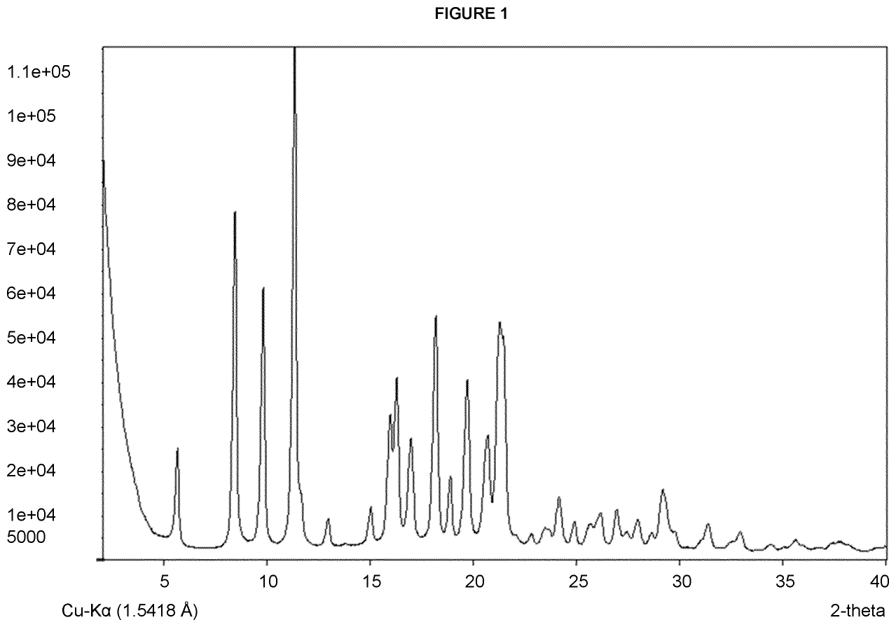

Even more preferably said stable crystalline form of tezacaftor is characterized by an XRPD profile substantially as shown by FIG. 1.

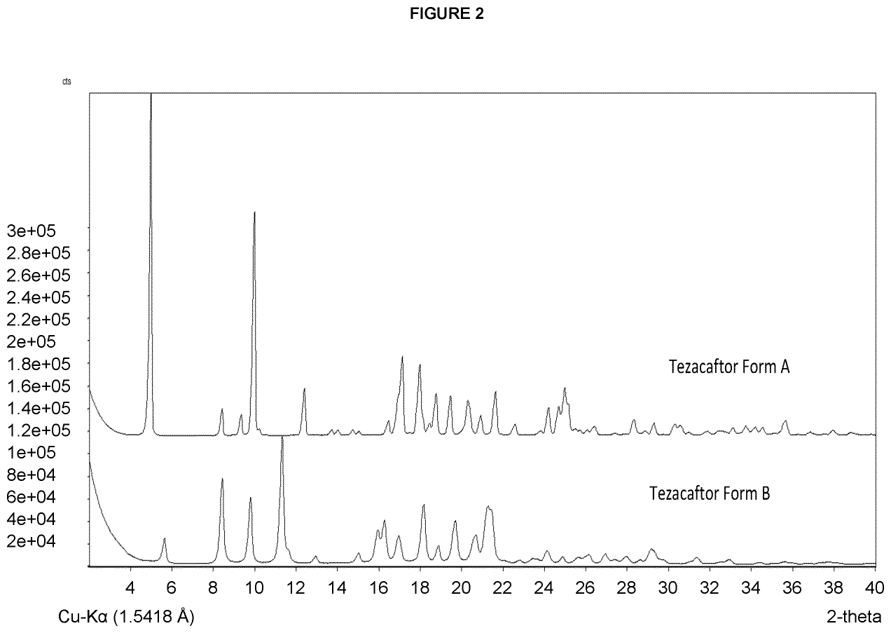

FIG. 2 shown a comparison of the XRPD profiles of the crystalline form B of tezacaftor according to the present invention and of the crystalline form A of tezacaftor according to WO 2011/119984, wherein the differences between the two forms are evident.

Preferably the polymorphically and/or chemically stable crystalline form of tezacaftor according to the present invention is characterized by a differential scanning calorimetry (DSC) thermogram at a heating rate of 10° C./min comprising a melting endotherm at 110-128° C., more preferably with a peak at 118° C.

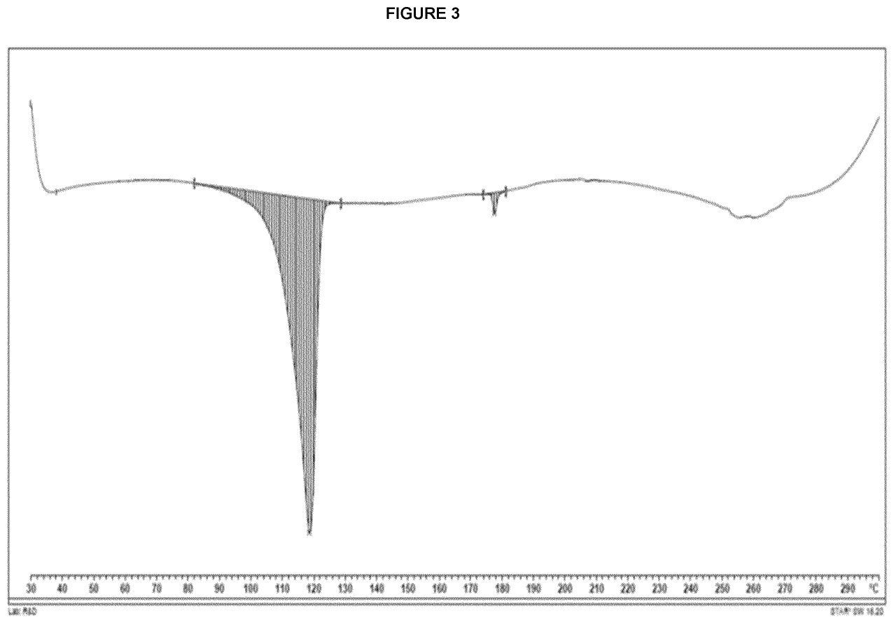

An exemplary DSC thermogram of the crystalline form of tezacaftor according to the present invention is shown in FIG. 3.

It was found that crystalline form B according to the present invention is advantageously stable, both chemically and from the crystalline point of view, as shown by the data in the experimental part, relative to accelerated (stressed) stability tests and is particularly suitable for storage and/or formulation for the preparation of pharmaceutical dosage forms.

In a preferred embodiment, the present invention relates to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor) in solid form B as described above, substantially free of any amorphous and other crystalline form, i.e. wherein no solid form other than form B, as above described, can be detected within the limits of suitable analytical methods and instruments, such as, but not limited to, XRD, DSC or TGA analysis.

In another aspect, the present invention relates to a process for the preparation of the polymorphically and/or chemically stable crystalline form B of tezacaftor, that is easily reproducible and scalable for production up to industrial scale, with the minimal adjustments known to the person skilled in the art, if required.

The process for the preparation of the polymorphically and/or chemically stable crystalline form B of tezacaftor according to the present invention comprises the following steps:
a) providing a solution of tezacaftor in at least one organic solvent;
b) bringing into contact the solution of tezacaftor provided in step a) with an antisolvent so as to cause its precipitation in the crystalline form B; and
c) isolating and optionally drying the solid obtained in step b),
   wherein at least one organic solvent in step a) is selected from the group consisting of a C1-C8 linear or branched alkyl alcohol, a C4-C10 linear or cyclic aliphatic or aromatic ether, a C3-C10 linear or branched alkyl ketone, and mixtures thereof, and the antisolvent in step b) is selected from the group consisting of a C5-C15 linear or branched alkane, a C6-10 aromatic solvent, and mixtures thereof.

Preferably step a) comprises providing a solution of tezacaftor in at least one organic solvent at a temperature from 10° C. to 35° C., more preferably from 15° C. to 30° C., even more preferably from 20° C. to 25° C.

Preferably the solution of tezacaftor provided in step a) is prepared according to the following steps:
a.1) bringing tezacaftor into contact with the at least one organic solvent so as to obtain a mixture; and
a.2) maintaining under stirring the resulting mixture so as to obtain a solution of tezacaftor in the at least one organic solvent.

The volume of the at least one organic solvent used in step a) is normally from 1 mL to 50 mL per gram of tezacaftor. Preferably said volume of the at least one organic solvent is between and optionally includes any two of the following values: 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL or 45 mL per gram of tezacaftor. More preferably this volume is from 4 mL to 25 mL per gram of tezacaftor.

The following step a.2) can be carried out according to either step a.2.1) or the sequence of steps a.2.2) and a.2.3), as defined hereunder.

Step a.2.1) preferably comprises maintaining the mixture obtained in step d.1) under stirring preferably at a temperature from 10 to 35° C. (more preferably from 15° C. to 28° C., even more preferably from 20° C. to 25° C.) until a solution of tezacaftor in the at least one organic solvent is obtained.

Alternatively, according to step a.2.2), the method preferably comprises heating the mixture obtained in step a.1) to a temperature from 36° C. to 80° C. (more preferably from 40° C. to 75° C., even more preferably from 50 to 65° C.) so as to provide a solution of tezacaftor in the at least one organic solvent. Preferably, according to step a.2.3), said solution is subsequently cooled to a temperature from 10° C. to 35° C., more preferably from 12° C. to 28° C., even more preferably from 15° C. to 25° C.

A preferred variant of the process object of this aspect of the invention comprises an additional and optional step a′), carried out after step a), a.2), a.2.1) or a.2.3), said step a′) comprising removing any undissolved particles from the solution obtained in step a), a.2), a.2.1), a.2.2) or a.2.3) (preferably by filtration, optionally under reduced pressure).

The following step b) comprises mixing the solution of tezacaftor prepared in step a), a.2), a.2.1), a.2.2) or a.2.3), optionally after having performed step a′), with an antisolvent, that is a liquid wherein tezacaftor is practically insoluble, preferably an antisolvent miscible with the organic solvent used in step a) at 20-25° C., more preferably wherein the antisolvent is selected from water or an aliphatic hydrocarbon, either cyclic or acyclic, and mixtures thereof, even more preferably an antisolvent selected from the group consisting of water, hexane, cyclohexane and n-heptane), so as to precipitate at least a portion of tezacaftor as a crystalline solid. The antisolvent addition changes the equilibrium solubility of tezacaftor in the solution prepared in step a), a.2), a.2.1), a.2.3) or a′) such that its concentration is supersaturated (i.e. above its equilibrium solubility limit) and tezacaftor subsequently precipitates from the solution. Particularly useful antisolvents are those in which tezacaftor is sparingly soluble, such as those in which this compound is soluble in amounts of not more than about 0.5% by weight at 20-25° C., preferably of not more than 0.25% by weight (more preferably of not more than 0.1% by weight) at 20-25° C.

The volume of the antisolvent is normally from 5 mL to 100 mL per gram of tezacaftor. Preferably the volume of the antisolvent is between and optionally includes any two of the following values: 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL, 50 mL, 55 mL, 60 mL, 65 mL, 70 mL, 75 mL, 80 mL, 85 mL, 90 mL or 95 mL per gram of tezacaftor.

The volume ratio between the antisolvent used in step b) and the at least one organic solvent used in step a) is typically from 30:1 to 100:1, preferably from 35:1 to 80:1, more preferably from 40:1 to 70:1, even more preferably from 45:1 to 60:1, for example from 48:1 to 55:1.

According to a preferred embodiment of this aspect of the invention, the solution of tezacaftor prepared in step A), a.2), a.2.1), a.2.3) or a′) is maintained at a temperature of at maximum 15° C., preferably at a temperature of at maximum 10° C., more preferably at a temperature of at maximum 5° C. when brought into contact with the antisolvent.

In a preferred embodiment of the present invention, in step b) of the process the solution of tezacaftor provided in step a) is added to the antisolvent so as to obtain precipitation of tezacaftor form B.

According to a further preferred embodiment of this aspect of the invention, the solution of tezacaftor prepared in step a), a.2), a.2.1), a.2.3) or a′) is added to the antisolvent (more preferably maintained at a temperature of at maximum 15° C., preferably at a temperature of at maximum 10° C., more preferably at a temperature of at maximum 5° C.).

The direct addition of the antisolvent to the solution of tezacaftor prepared in step a), a.2), a.2.1), a.2.3) or a′), i.e. with addition of the solution of tezacaftor to the antisolvent, or the inverse addition of the antisolvent to any one of these solutions can be carried out in a single step (i.e. a single addition of the entire volume of the solvent or solution to be added) or, alternatively, in multiple additions. Preferably the solution of tezacaftor prepared in step a), a.2), a.2.1), a.2.3) or a′) is added to the antisolvent dropwise.

In a further variant of the process object of this aspect of the invention, an additional and optional step b′) is carried after step b), in which the suspension obtained in step b) is maintained under stirring (preferably at a temperature of at maximum 15° C., more preferably at a temperature of at maximum 10° C., even more preferably at a temperature of at maximum 5° C.) so as to increase the precipitation rate of tezacaftor.

After precipitation, tezacaftor is recovered in step c) using known techniques such as filtration or centrifugation and optionally dried, e.g. according to the any of the procedures known in the field, preferably by treating the resulting solid at a temperature from 30° C. to 70° C. (more preferably from 35° C. to 65° C., even more preferably from 40° C. to 50° C.) optionally under reduced pressure.

“C4-C10 linear, branched or cyclic aliphatic or aromatic ether” indicates any organic solvent comprising at least one C—O—C moiety and having a molecular formula comprising from 4 to 10 carbon atoms, with or without an aromatic moiety according to the definition of the IUPAC gold book. Non-limiting examples of such solvents are diethyl ether, diisopropyl ether, dipropyl ether, methyl tert-butyl ether, tetrahydrofuran (THF), 2- and 3-methyl THF, dioxane, dimethoxyethane and similar glycols, anisole and analogues thereof.

Non-limiting examples of C1-C8 linear or branched alkyl alcohols are methanol, ethanol, n-and iso-propanol, butanol, pentanol, hexanol and all isomers thereof.

Non-limiting examples of C3-C10 linear or branched alkyl ketones are acetone, methyl ethyl ketone, methyl propyl ketone, methyl isobutyl ketone and analogues thereof.

In a preferred embodiment, the at least one organic solvent is at least one linear or cyclic aliphatic ether, preferably selected from the group consisting of diethyl ether, tetrahydrofuran, 2-methyltetrahydrofuran, 3-methyltetrahydrofuran, and mixtures thereof, more preferably 2-methyltetrahydrofuran.

Similarly, “a C5-C15 linear or branched alkane” indicates alkyl solvents comprising from 5 to 15 carbon atoms, such as, without limitation, pentane, cyclopentane, hexane, iso-hexane, cyclohexane, linear or branched heptane, octane and analogues, and “a C6-10 aromatic solvent” indicates a solvent comprising a benzene moiety, such as, without limitation, benzene, toluene, xylene, ethylbenzene and analogues.

In a preferred embodiment of the present invention, the antisolvent used in step b) is an aliphatic hydrocarbon, preferably selected from the group consisting of pentane, hexane, cyclohexane, heptane, octane, and mixtures thereof.

The stable crystalline form of tezacaftor of the present invention may be administered to a subject in the need thereof in the form of the pharmaceutical composition alone. Alternatively, said stable crystalline form of tezacaftor can be provided in the form of a medicament (e.g., a pharmaceutical formulation) comprising such form in combination with at least one component selected from the group comprising (preferably consisting of) a pharmaceutically acceptable carrier, an excipient, a disintegrator, a binder, a fluidizing agent, a diluent, a filler, a buffer, an adjuvant, a stabilizer, a preservative, a lubricant, a solvent, a solubilizer, a suspending agent, an isotonizing agent, a soothing agent and other materials known in the art, and, according to need, other drugs (e.g. ivacaftor and/or elexacaftor).

Examples of forms of pharmaceutical formulations (dosage forms) include, but are not limited to, oral formulations, such as tablets, capsules, granules, powders, troches, syrups, emulsions, and suspensions.

In an aspect, the present invention relates to a pharmaceutical formulation comprising (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor) in the crystalline form B, preferably wherein the pharmaceutical composition is an oral dosage form selected from the group consisting of tablet, capsule, granules, powder, troche, syrup agents, emulsions, and suspensions.

The present invention also provides for a method for producing a pharmaceutical formulation comprising mixing the stable crystalline form of tezacaftor of the present invention with a pharmaceutically acceptable carrier or excipient, particularly selected from the group consisting of a disintegrator, a binder, a fluidizing agent, a diluent, a filler, a buffer, an adjuvant, a stabilizer, a preservative, a lubricant, a solvent, a solubilizer, a suspending agent, an isotonizing agent, and a soothing agent, and, optionally, one or more other drugs (e.g. ivacaftor and/or elexacaftor).

In an aspect, the present invention is relative to the use of said stable crystalline form B of tezacaftor for preparing other solid-state forms of tezacaftor, preferably form A or amorphous tezacaftor.

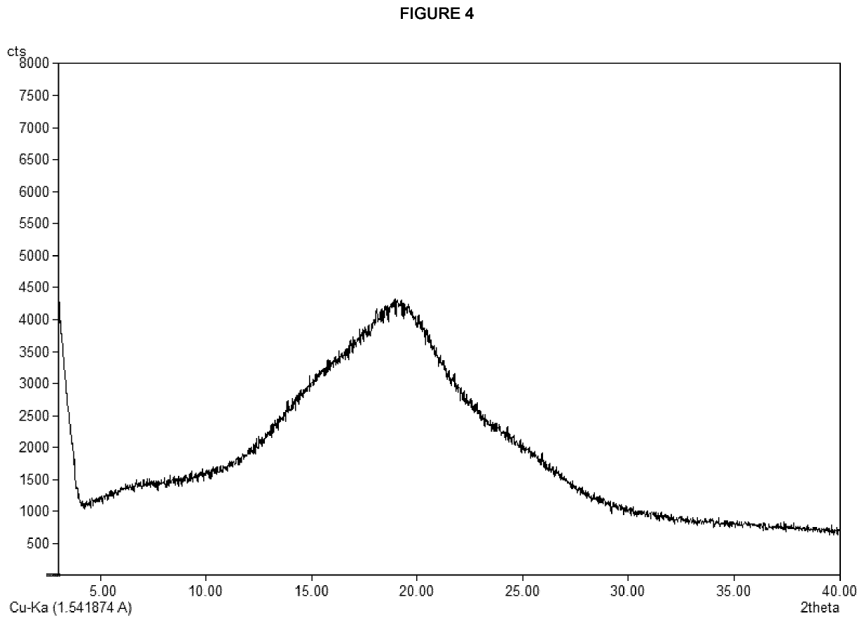

In another aspect, the present invention relates to a polymorphically and/or chemically stable amorphous form of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor), preferably a polymorphically and chemically stable amorphous form of tezacaftor, said stable amorphous form being more preferably characterized by an XRPD profile comprising a halo pattern between 8 and 33° 2θ when collected with the Kα radiation of copper (λ=1.5418 λ). Even more preferably said stable amorphous form of tezacaftor is characterized by an XRPD profile substantially as illustrated by FIG. 4. Preferably the polymorphically and/or chemically stable amorphous form of tezacaftor object of the first aspect of the invention has a maximum water content, as determined by K.F. titration, of 8% by weight. According to a more preferred embodiment of this aspect of the invention, said polymorphically and/or chemically stable amorphous form has a maximum water content, as determined by K.F. titration, of 7% by weight, for example lower than 5%, 4%, 3%, 2% or 1% by weight.

Another aspect of the present invention relates to two alternative processes for the preparation of the polymorphically and/or chemically stable amorphous form of tezacaftor, which, in the following will be referred to simply as first and second method.

The first method comprises the following steps:
a) providing a solution of tezacaftor in at least one organic solvent or in a mixture comprising at least one organic solvent and water;
b) freeze-drying the solution obtained in step a); and
c) isolating and optionally drying the resulting solid.

According to step a) a solution of tezacaftor in at least one organic solvent (preferably a water miscible organic solvent) or in a mixture comprising at least one organic solvent and water is provided. Preferably this step is carried out at a temperature from 10 to 40° C., more preferably from 15° C. to 30° C., even more preferably from 20° C. to 25° C.

Tezacaftor suitable for the purposes of the present invention is commercially available; alternatively, it can be prepared according to techniques known in organic synthesis, e.g., according to the procedures described in US 2009/0131492 or WO 2011/119984 A1.

Organic solvents (preferably water miscible organic solvents) useful for the aim are generally known in the field, and are preferably selected from the group consisting of alcohols (preferably a C1-C4 alcohol, for example methanol, ethanol, isopropanol, propanol, butanol, isobutanol, sec-butanol and tert-butanol), ketones (either cyclic or acyclic, preferably a C3-C6 ketone, e.g., acetone, 4-methyl-2-pentanone, 2-butanone), ethers, (either cyclic or acyclic, such as dioxane, tetrahydrofuran, 2-methyltetrahydrofuran, methyl tert-butyl ether), polar aprotic solvents (such as dimethylformamide, dimethylsulfoxide and dimethylacetamide) and mixtures thereof.

The volume of the liquid used in step a) (being either an organic solvent or a mixture thereof with water) can vary in a very wide range; preferably, said volume of liquid is from 10 mL to 100 mL per gram of tezacaftor. More preferably said volume of liquid is between and optionally includes any two of the following values: 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 35 mL, 40 mL, 45 mL, 50 mL, 55 mL, 60 mL, 65 mL, 70 mL, 75 mL, 80 mL, 85 mL, 90 mL or 95 mL per gram of tezacaftor. Even more preferably said volume of liquid is from 20 mL to 50 mL per gram of tezacaftor, e.g., 40 mL per gram of tezacaftor.

In the subsequent step b) the solution obtained in step a) is freeze dried (lyophilized) according to any one of the methods known in the field and comprising, for example, the freezing of a solution followed by a reduction of the pressure to remove the solvent. Conditions suitable for freezing the solutions of step a), depending on the solvent chosen to prepare said solution, may entail a temperature from −80° C. to 0° C. (preferably from −50° C. to −25° C., for example from −45° C. to −35° C.) preferably under atmospheric pressure (i.e. about 1 bar). The removal of the solvent from the frozen solution may include treating it to a temperature from −35° C. to 15° C. (preferably from −30° C. to 5° C., for example from −25° C. to 0° C.) at a pressure preferably from 0.01 to 1 mbar (more preferably 0.1 mbar). Even more preferably the removal of the solvent from the frozen solution is performed by subsequently treating it to (i) a temperature from −35° C. to −15° C. (preferably from −30° C. to −20° C., more preferably at −25° C.) and a pressure preferably from 0.01 to 1 mbar (more preferably 0.1 mbar), (ii) a temperature from −15° C. to −5° C. (preferably at −10° C.) and a pressure preferably from 0.01 to 1 mbar (more preferably 0.1 mbar), and (iii) a temperature from −5° C. to 0° C. and a pressure preferably from 0.01 to 1 mbar (more preferably 0.1 mbar).

Even more preferably, in step (i) the temperature is maintained from −35° C. to −15° C. for at least 5 hours (preferably for at least 10 hours, more preferably for at least 15 hours), in step (ii) the temperature is maintained from −15° C. to −5° C. for at least 1 hour (preferably for at least 2 hours, more preferably for at least 3 hours, even more preferably for at least 4 hours) and in step (iii) the temperature is maintained from −5° C. to 0° C. for at least 1 hour (preferably for at least 2 hours, more preferably for at least 3 hours, even more preferably for at least 4 hours).

If the organic solvent used in step a) is not compatible with the freeze-drying process performed in step b), a variant of the first method includes an additional step a), carried out after step a), in which said solvent is evaporated, preferably under reduced pressure. If necessary, the mass obtained after distillation of the organic solvent is diluted (preferably with a water miscible organic solvent) so as to obtain a solution, which can be lyophilized.

The resulting amorphous form of tezacaftor is isolated in step c) and optionally dried, e.g. according to the any of the procedures known in the field, preferably by treating the solid at a temperature from 30° C. to 70° C. (more preferably from 35° C. to 65° C., even more preferably from 40° C. to 50° C.) optionally under reduced pressure.

The second method for the preparation of the polymorphically and/or chemically stable amorphous form of tezacaftor comprises the following steps:
d) providing a solution of tezacaftor in at least one organic solvent;
e) bringing into contact the solution of tezacaftor provided in step d) with an antisolvent so as to cause its precipitation in the form of an amorphous powder; and
f) isolating and optionally drying the resulting solid.

Step d) comprises providing a solution of tezacaftor in at least one organic solvent preferably at a temperature from 10° C. to 35° C., more preferably from 15° C. to 30° C., even more preferably from 20° C. to 25° C.

Preferably the solution of tezacaftor provided in step d) is prepared according to the following steps:
d.1) bringing into contact tezacaftor with the least one organic solvent so as to obtain a mixture; and
d.2) maintaining under stirring the resulting mixture so as to obtain a solution of tezacaftor in the at least one organic solvent.

Step d.1) comprises bringing into contact tezacaftor with the least one organic solvent so as to obtain a mixture.

Organic solvents suitable to be used in step d) or d.1) are generally known in the field, and are preferably selected from the group consisting of alcohols (preferably C1-C4 alcohols, for example methanol, ethanol, or preferably tert-butanol), ketones (e.g. acetone), esters (preferably ethyl acetate), aromatic hydrocarbons (preferably toluene), chlorinated solvents (preferably dichloromethane), ethers (either cyclic or acyclic, preferably tetrahydrofuran or dioxane) and mixtures thereof.

The volume of the at least one organic solvent used in step d) or d.1) is normally from 1 mL to 50 mL per gram of tezacaftor. Preferably said volume of the at least one organic solvent is between and optionally includes any two of the following values: 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL or 45 mL per gram of tezacaftor. More preferably this volume is from 4 mL to 25 mL per gram of tezacaftor.

The following step d.2) can be carried out according to either step d.2.1) or the sequence of steps d.2.2) and d.2.3).

Step d.2.1) comprises maintaining the mixture obtained in step d.1) under stirring preferably at a temperature from 10 to 35° C. (more preferably from 15° C. to 28° C., even more preferably from 20° C. to 25° C.) until a solution of tezacaftor in the at least one organic solvent is obtained.

The alternative synthetic path includes heating the mixture obtained in step d.1) to a temperature preferably from 36° C. to 80° C. (more preferably from 40° C. to 75° C., even more preferably from 50 to 65° C.) so as to provide a solution of tezacaftor in the at least one organic solvent, according to step d.2.2). Said solution is subsequently cooled to a temperature preferably from 10 to 35° C., more preferably from 12° C. to 28° C., even more preferably from 15° C. to 25° C., according to step d.2.3).

A variant of the process object of this aspect of the invention includes an additional and optional step d′), carried out after step d), d.2), d.2.1), d.2.2) or d.2.3), said step d′) comprising removing any undissolved particles from the solution obtained in step d), d.2), d.2.1), d.2.2) or d.2.3) (preferably by filtration, optionally under reduced pressure).

The following step e) includes mixing the solution of tezacaftor prepared in step d), d.2), d.2.1) or d.2.3), optionally after having performed step d′), with an antisolvent (preferably an antisolvent miscible with the organic solvent used in step d), more preferably water or an aliphatic hydrocarbon either cyclic or acyclic, even more preferably an antisolvent selected from the group consisting of water, hexane, cyclohexane and n-heptane) so as to precipitate at least a portion of tezacaftor as an amorphous powder. The antisolvent functions to change the equilibrium solubility of tezacaftor in the solution prepared in step d), d.2), d.2.1), d.2.3) or d′) such that its concentration is supersaturated (i.e. above its equilibrium solubility limit and it precipitates from the solution. Useful antisolvents are those in which tezacaftor is sparingly soluble, such as those in which this compound is soluble in amounts of not more than about 0.5% by weight at 20-25° C., preferably of not more than 0.25% by weight (more preferably of not more than 0.1% by weight) at 20-25° C.

The volume of the antisolvent is normally from 5 mL to 100 mL per gram of tezacaftor. Preferably the volume of the antisolvent is between and optionally includes any two of the following values: 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL, 50 mL, 55 mL, 60 mL, 65 mL, 70 mL, 75 mL, 80 mL, 85 mL, 90 mL or 95 mL per gram of tezacaftor.

The volume ratio between the antisolvent and the at least one organic solvent used in step d) or d.1) is typically from 1:1 to 25:1, preferably from 2:1 to 20:1, more preferably from 3:1 to 15:1, even more preferably from 4:1 to 12:1, for example from 5:1 to 10:1.

According to a preferred embodiment of this aspect of the invention, the solution of tezacaftor prepared in step d), d.2), d.2.1), d.2.3) or d′) is brought into contact with the antisolvent maintained at a temperature of at maximum 15° C., preferably at a temperature of at maximum 10° C., more preferably at a temperature of at maximum 5° C.

According to a further preferred embodiment of this aspect of the invention, the solution of tezacaftor prepared in step d), d.2), d.2.1), d.2.3) or d′) is added to the antisolvent (more preferably maintained at a temperature of at maximum 15° C., preferably at a temperature of at maximum 10° C., more preferably at a temperature of at maximum 5° C.).

The direct addition of the antisolvent to the solution of tezacaftor prepared in step d), d.2), d.2.1), d.2.3) or d′), or the inverse addition of any one of these solutions to the antisolvent can be carried out in a single step (i.e. a single addition of the entire volume of the solvent or solution to be added) or, alternatively, in multiple additions. Preferably the solution of tezacaftor prepared in step d), d.2), d.2.1), d.2.3) or d′) is added to the antisolvent dropwise.

In a further variant of the process object of this aspect of the invention, an additional and optional step e′) is carried after step e), in which the suspension obtained in step e) is maintained under stirring (preferably at a temperature of at maximum 15° C., more preferably at a temperature of at maximum 10° C., even more preferably at a temperature of at maximum 5° C.) so as to increase the precipitation rate of tezacaftor.

After precipitation, tezacaftor is recovered in step f) using known techniques such as filtration or centrifugation and optionally dried, e.g. according to the any of the procedures known in the field, preferably by treating the resulting solid at a temperature from 30° C. to 70° C. (more preferably from 35° C. to 65° C., even more preferably from 40° C. to 50° C.) optionally under reduced pressure.

The stable amorphous form of tezacaftor of the present invention may be administered to a subject in the need thereof in the form of the pharmaceutical composition alone.

Alternatively, said stable amorphous form of tezacaftor can be provided in the form of a medicament (e.g., a pharmaceutical formulation) comprising such compound in combination with at least one member selected from the group comprising (preferably consisting of) a pharmaceutically acceptable carrier, an excipient, a disintegrator, a binder, a fluidizing agent, a diluent, a filler, a buffer, an adjuvant, a stabilizer, a preservative, a lubricant, a solvent, a solubilizer, a suspending agent, an isotonizing agent, a soothing agent and other materials known in the art, and, according to need, other drugs (e.g. ivacaftor and/or elexacaftor).

Examples of forms of pharmaceutical formulations (dosage forms) include, but are not limited to, oral formulations, such as tablets, capsules, granules, powders, troches, syrup agents, emulsions, and suspending agents.

The present invention also provides for a method for producing a pharmaceutical formulation comprising mixing the stable amorphous form of tezacaftor of the present invention with a pharmaceutically acceptable carrier, an excipient, a disintegrator, a binder, a fluidizing agent, a diluent, a filler, a buffer, an adjuvant, a stabilizer, a preservative, a lubricant, a solvent, a solubilizer, a suspending agent, an isotonizing agent, a soothing agent and/or other materials known in the art, and, according to need, other drugs (e.g. ivacaftor and/or elexacaftor).

### Experimental Part

XRPD analyses were performed at 20-30° C. by means of a theta/theta vertical scan Bruker AXS D8 Advance high-performance diffractometer. The instrument was equipped with a Cu-Kα X-ray tube operating at 40 kV/40 mA and generating radiation having λ=1.5418 Å. The detector was a linear Lynxeye XE-T position sensitive set at 250 mm from the sample. Powder samples were deposited in the 20 mm×0.5 mm hollow of the sample holder, consisting of a quartz monocrystal zero background plate. A mild grinding of the sample in an agate mortar may be needed to obtain a suitable fine powder. Diffraction data were collected applying the following conditions: angular range 2-40° 2-theta, 0.02°/step scan and 1 second acquisition time. The instrument calibration was verified by means of a NIST SRM 1976b. Data acquisition was performed by means of Bruker Diffraction Measurement Center software. Data elaboration was performed by means of Crystal Impact Match!.

DSC analyses were carried out with a Mettler DSC-1 Star System, using 40 μL standard aluminium crucibles loaded with about 4-7 mg of sample and sealed with a specific crucible sealing press. A void crucible of the same kind was placed in the reference location of the DSC furnace. Indium reference material was used to calibrate the apparatus with regard to the temperature scale and the enthalpy response. The samples were analysed under nitrogen flow at a heating rate of 10° C./min after making a pinhole on the crucible lid. Onset and peak temperatures (° C.) and ΔH of the measured transitions (J/g) were generally considered parameters of interest. The explored thermal range was between 30° C. and up to 250° C.

### EXAMPLES

### Example 1

### Preparation of Crystalline Tezacaftor by Solvent/Antisolvent Procedure.

In a 250 mL three-neck round bottom flask equipped with magnetic stirrer, thermometer and kept under nitrogen atmosphere are charged 32.2 g of tezacaftor and 104 mL of 2-MeTHF. The suspension is heated to reflux (80° C.), then it is allowed to cool to 20-25° C.

The solution is filtered, the filter is washed with 15 mL of 2-MeTHF and the resulting solution is transferred in a dropping funnel.

In a second four-neck round bottom flask equipped with mechanical stirrer, thermometer and kept under nitrogen atmosphere are charged 1193 mL of n-heptane and it is cooled to 0° C.

The solution of tezacaftor in 2-MeTHF is added dropwise in not less than 1 hour to the 1193 mL of n-heptane at 0° C. in the second four-neck round bottom flask.

The suspension is stirred at 0° C. for 1 hour, then it is filtered on a Buchner funnel and the solid is washed two times with a mixture of 2-MeTHF/n-heptane 1:10 respectively.

The wet solid (37 g) is dried under vacuum at 20-25° C. for 16 hours and then at 40° C. under vacuum for 16 hours.

28.4 g of tezacaftor form B are obtained.

HPLC purity 99.66%, no peaks with area >0.1%.

Comparable results were obtained using n-hexane as the antisolvent.

An aliquot of the solid was analysed by XRPD, obtaining a diffractogram corresponding to that shown in FIG. 1 (crystal form B).

The following table shows the diffraction peaks of form B according to the present invention:

| No. | 2theta [°] | d [Å] | I/I0 |
|---|---|---|---|
| 1 | 3.65 | 241.861 | 8.51 |
| 2 | 5.63 | 157.041 | 162.31 |
| 3 | 8.42 | 105.005 | 629.45 |
| 4 | 9.79 | 90.377 | 472.88 |
| 5 | 11.31 | 78.222 | 1000.00 |
| 6 | 11.63 | 76.106 | 91.91 |
| 7 | 12.93 | 68.467 | 47.78 |
| 8 | 13.77 | 64.323 | 3.59 |
| 9 | 15.00 | 59.074 | 63.00 |
| 10 | 15.94 | 55.587 | 238.95 |
| 11 | 16.26 | 54.511 | 314.36 |
| 12 | 16.96 | 52.276 | 196.40 |
| 13 | 18.16 | 48.843 | 443.63 |
| 14 | 18.86 | 47.054 | 100.55 |
| 15 | 19.68 | 45.106 | 314.75 |
| 16 | 20.66 | 43.001 | 205.09 |
| 17 | 21.30 | 41.706 | 468.84 |
| 18 | 21.43 | 41.464 | 416.90 |
| 19 | 22.01 | 40.378 | 9.98 |
| 20 | 22.78 | 39.030 | 16.56 |
| 21 | 23.48 | 37.888 | 29.85 |
| 22 | 23.65 | 37.624 | 26.89 |
| 23 | 24.13 | 36.879 | 90.21 |
| 24 | 24.87 | 35.805 | 39.80 |
| 25 | 25.64 | 34.743 | 36.28 |
| 26 | 26.13 | 34.102 | 56.71 |
| 27 | 26.94 | 33.095 | 59.43 |
| 28 | 27.41 | 32.539 | 16.47 |
| 29 | 27.95 | 31.919 | 42.83 |
| 30 | 28.63 | 31.181 | 18.09 |
| 31 | 29.19 | 30.592 | 108.00 |
| 32 | 29.68 | 30.103 | 28.18 |
| 33 | 30.92 | 28.919 | 8.34 |
| 34 | 31.36 | 28.527 | 46.41 |
| 35 | 32.46 | 27.584 | 12.82 |
| 36 | 32.91 | 27.218 | 33.60 |
| 37 | 34.40 | 26.072 | 8.76 |
| 38 | 35.03 | 25.619 | 2.65 |
| 39 | 35.61 | 25.213 | 16.36 |
| 40 | 35.94 | 24.988 | 8.04 |
| 41 | 36.70 | 24.489 | 2.25 |
| 42 | 37.38 | 24.058 | 9.29 |
| 43 | 37.71 | 23.855 | 12.79 |
| 44 | 38.14 | 23.597 | 8.08 |
| 45 | 39.60 | 22.761 | 1.95 |

The sample was analysed by DSC at a heating rate of 10° C./min, as described above, and a thermogram comprising a melting endotherm at 110-128° C. (peak at 118° C.) was observed (shown FIG. 3).

### Comparative Example 1.1

For comparison, form A was prepared according the procedure of WO 2011/119984 and analysed by XRPD, obtaining the diffractogram shown in the upper part of FIG. 2. The XRPD diffractogram of crystal form B of tezacaftor according to the present invention in shown in the lower part of FIG. 2.

The DSC thermogram of form A, following the method as described above at a heating rate of 10° C./min, showed a melting endotherm with a peak at 179° C.

### Example 2

### Analysis of the Stressed Stability of the Crystalline Tezacaftor Form Prepared According to the Processes of the Invention.

### Example 2.1

The crystalline tezacaftor, prepared as described in Example 1, was maintained at 20-25° C. at 80% relative humidity (RH) in an open vial for 4 weeks. The solid was then subjected to XRPD analysis, showing a XRPD diffractogram corresponding to the one obtained in example 1.

### Example 2.2

The crystalline tezacaftor, prepared as described in Example 1, was maintained, in an open vial, at 40° C. and 75% RH for 4 weeks. The solid was then brought to room temperature and subjected to XRPD analysis and showed a XRPD diffractogram corresponding to that obtained in example 1.

### Example 2.3

The crystalline tezacaftor, prepared as described in Example 1, was maintained, in a closed vial, at 60° C. for 4 weeks. The solid was then brought to room temperature and subjected to XRPD analysis and the obtained XRPD diffractogram corresponded to that obtained from the sample of example 1.

The samples of examples 3.1-3.3 at the end of the stressed stability test were analysed by HPLC. No detectable increase in any impurity was found in any of the samples.

### Example 3

### Preparation of Amorphous Tezacaftor by Freeze-Drying.

Tezacaftor (5.0 g) was dissolved, under magnetic stirring and at 25° C., in a mixture comprising tert-butanol (100 mL) and water (100 mL). The resulting solution was freeze-dried according to the following program and ground to obtain an amorphous solid characterized by an XRPD spectrum as depicted in FIG. 4.

| Step | Time | | Pressure (mbar) | Temperature | |
|---|---|---|---|---|---|
| Pre-freezing | 35 | hours | 1000 | −35° | C. |
| Primary Drying Step 1 | 16 | hours | 0.1 | −25° | C. |
| Primary Drying Step 2 | 5 | hours | 0.1 | −10° | C. |
| Primary Drying Step 3 | 4 | hours | 0.1 | −5° | C. |
| Secondary Drying Step 1 | 15 | hours | 0.1 | 5° | C. |
| Secondary Drying Step 2 | 1 | hour | 0.1 | 15° | C. |

### Example 4

### Preparation of Amorphous Tezacaftor by Solvent/Antisolvent Procedure.

Tezacaftor (5.0 g) was suspended in methanol (20 mL), under magnetic stirring and at 25° C. The suspension was heated under stirring to 60° C., and maintained at the same temperature until a solution was obtained (about 1 hour). The solution was cooled to 25° C. and dripped into demineralised water (100 mL) previously cooled to 0/5° C. (using an ice bath), monitoring that the internal temperature did not exceed 5° C. The resulting mixture was maintained under stirring at the same temperature for 30 minutes then it was filtered. The solid was washed with demineralised water (cooled to 0/5° C.) and dried at 40° C. under reduced pressure, thus affording 4.52 grams of tezacaftor as a solid.

An aliquot of the solid was analysed by XRPD, obtaining a diffractogram corresponding to that shown in FIG. 4.

### Example 5

### Preparation of Amorphous Tezacaftor by Solvent/Antisolvent Procedure.

Tezacaftor (5.4 g) was suspended in tetrahydrofuran (20 mL), under magnetic stirring and at 25° C. The suspension was heated under stirring to 60° C., and maintained at the same temperature until a solution was obtained (about 1 hour). The solution was cooled to 25° C. and dripped into n-heptane (200 mL) previously cooled to 0° C., monitoring that the internal temperature did not exceed 5° C. The resulting mixture was maintained under stirring at the same temperature for 60 minutes then it was filtered. The solid was washed with n-heptane (cooled to 0° C.) and dried at 40° C. under reduced pressure, thus affording 4.88 grams of tezacaftor as a solid.

An aliquot of the solid was analysed by XRPD, obtaining a diffractogram corresponding to that shown in FIG. 4.

### Example 6

### Analysis of the Stability of the Amorphous Tezacaftor Prepared According to the Processes of the Invention.

### Example 6.1

The amorphous tezacaftor, prepared as described in Examples 3 to 5, was maintained at 70° C. under a reduced pressure for 12 hours. The solid was then brought to room temperature and subjected to XRPD analysis, giving rise to a XRPD spectrum corresponding to the one obtained in example 3.

### Example 6.2

The amorphous tezacaftor, prepared as described in Examples 3 to 5, was maintained, in a closed vial, at 60° C. for 15 days. The solid was then brought to room temperature and subjected to XRPD analysis, giving rise to a XRPD spectrum corresponding to the one obtained in example 3.

### Example 6.3

The amorphous tezacaftor, prepared as described in Examples 3 to 5, was maintained, in an open vial, at 40° C. and 75% RH for 15 days. The solid was then brought to room temperature and subjected to XRPD analysis, giving rise to a XRPD spectrum corresponding to the one obtained in example 3.

### Example 6.4

The amorphous tezacaftor, prepared as described in Examples 3 to 5, was maintained, in an open vial, at 25-30° C. and 80% RH for 15 days. The solid was then brought to room temperature and subjected to XRPD analysis, giving rise to a XRPD spectrum corresponding to the one obtained in example 3.

The following paragraphs of the description refer to further aspects of the present disclosure, not presently claimed, listed as separate items:

1. Process for the preparation of a polymorphically and/or chemically stable amorphous form of tezacaftor, said process comprising the following steps:
a) providing a solution of tezacaftor in at least one organic solvent or in a mixture comprising at least one organic solvent and water;
b) freeze-drying the solution obtained in step a); and
c) isolating and optionally drying the resulting solid.

2. The process of item 1, in which the at least one organic solvent used in step a) comprises a water miscible organic solvent.

3. The process of any one of items 1 and 2, in which the at least one organic solvent used in step a) is a water miscible organic solvent.

4. The process of any one of items 1 to 3, in which the at least one organic solvent used in step a) is selected from the group consisting of an alcohol, a ketone, an ether, a polar aprotic solvent, and a mixture thereof.

5. The process of any one of items 1 to 4, in which the at least one organic solvent used in step a) is selected from the group consisting of a C1-C4 alcohol and a C3-C6 ketone.

6. The process of any one of items 1 to 5, in which the volume of liquid used in step a) is from 10 mL to 100 mL per gram of tezacaftor.

7. The process of any one of items 1 to 6, in which an additional step a′) is carried out after step a), said step a′) comprising evaporating the organic solvent used in step a) and optionally diluting the mass obtained after distillation so as to obtain a solution.

8. The process of item 7, in which the mass is diluted with at least one organic solvent.

9. The process of any one of items 1 to 8, in which step b) comprises freezing the solution of tezacaftor followed by a reduction of the pressure to remove the solvent.

10. The process of item 9, in which the freezing step comprises subjecting the solution of tezacaftor to a temperature from -80° C. to 0° C.

11. The process of any one of items 9 and 10, in which the removal of the solvent from the frozen solution comprises subjecting it to a temperature from -35° C. to 15° C. preferably at a pressure from 0.01 to 1 mbar.

12. The process of any one of items 9 to 11, in which the removal of the solvent from the frozen solution comprises subsequently subjecting it to:
(i) a temperature from -35° C. to -15° C. and a pressure preferably from 0.01 to 1 mbar;
(ii) a temperature from -15° C. to -5° C. and a pressure preferably from 0.01 to 1 mbar; and
(iii) a temperature from -5° C. to 0° C. and a pressure preferably from 0.01 to 1 mbar.

13. The process of item 12, in which in step (i) the temperature is maintained from −35° C. to −15° C. for at least 5 hours, in step (ii) the temperature is maintained from −15° C. to −5° C. for at least 1 hour and in step (iii) the temperature is maintained from −5° C. to 0° C. for at least 1 hour.

14. The process of any one of items 1 to 13, in which the amorphous form of tezacaftor is dried in step c) at a temperature from 30° C. to 70° C.

15. Polymorphically and/or chemically stable amorphous form of tezacaftor obtainable by the process of any one of items 1 to 15.

16. Polymorphically stable amorphous form of tezacaftor, said amorphous form showing no signs of crystallinity associated to crystalline tezacaftor when stored under any one of the following conditions (I) to (V):
(I) at 70° C. under reduced pressure for at least 1 hour; and/or
(II) at 60° C. for at least 1 day; and/or
(III) at 40° C. and 75% RH for at least 1 day; and/or
(IV) at 25-30° C. and 80% RH for at least 1 day; and/or
(V) a combination of any two or more (I)-(IV).

17. Chemically stable amorphous form of tezacaftor, said amorphous form showing no significant worsening of the purity profile when stored under any one of the following conditions (I) to (V):
(I) at 70° C. under reduced pressure for at least 1 hour; and/or
(II) at 60° C. for at least 1 day; and/or
(III) at 40° C. and 75% RH for at least 1 day; and/or
(IV) at 25-30° C. and 80% RH for at least 1 day; and/or
(V) a combination of any two or more (I)-(IV).

18. Polymorphically and chemically stable amorphous form of tezacaftor, said amorphous form showing no signs of crystallinity associated to crystalline tezacaftor and no significant worsening of the purity profile when stored under any one of the conditions (I) to (V):
(I) at 70° C. under reduced pressure for at least 1 hour; and/or
(II) at 60° C. for at least 1 day; and/or
(III) at 40° C. and 75% RH for at least 1 day; and/or
(IV) at 25-30° C. and 80% RH for at least 1 day; and/or
(V) a combination of any two or more (I)-(IV).

19. Polymorphically and/or chemically stable amorphous form of tezacaftor of any one of items 15 to 18, said stable amorphous form being characterized by an XRPD profile comprising a halo pattern between 8 and 33° 2θ when collected with the Kα radiation of copper.

20. Polymorphically and/or chemically stable amorphous form of tezacaftor of any one of items 15 to 19, said stable amorphous form having a maximum water content, as determined by K.F. titration, of at maximum 8% by weight.

21. Polymorphically and/or chemically stable amorphous form of tezacaftor of any one of items 15 to 20, said stable amorphous form having a maximum water content, as determined by K.F. titration, of at maximum 7% by weight.

22. Polymorphically and/or chemically stable amorphous form of tezacaftor of any one of items 15 to 21, said stable amorphous form having a water content, as determined by K.F. titration, lower than 5% by weight.

23. Polymorphically and/or chemically stable amorphous form of tezacaftor of any one of items 15 to 22 for use as a medicament.

24. Pharmaceutical composition comprising the polymorphically and/or chemically stable amorphous form of tezacaftor of any one of items 15 to 22.

25. Pharmaceutical composition comprising the polymorphically and/or chemically stable amorphous form of tezacaftor of any one of items 15 to 22 and at least one pharmaceutically acceptable carrier.

26. Pharmaceutical composition of any one of items 24 and 25 for use in medicine.

27. Use of the polymorphically and/or chemically stable amorphous form of tezacaftor of any one of items 15 to 22 for preparing other solid-state forms of tezacaftor.

28. Use of the polymorphically and/or chemically stable amorphous form of tezacaftor of any one of items 15 to 22 for preparing a crystalline form of tezacaftor.

29. Use of the polymorphically and/or chemically stable amorphous form of tezacaftor of any one of items 15 to 22 for preparing crystalline form A of tezacaftor.

1. A crystalline form of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor, Form B), characterized by an X-ray powder diffraction (XRPD) profile comprising at least peaks at 5.4-5.8, 11.1-11.5, 12.7-13.1 and 15.7-16.1 degrees 2θ, when collected with the Kα radiation of copper (λ=1.5418 Å).

2. The crystalline form of tezacaftor according to claim 1, characterized by an XRPD profile comprising at least the peaks at 5.6-5.7, 11.3-11.4, 12.8-12.9 and 15.8-16.0 degrees 2θ, when collected with the Kα radiation of copper (λ=1.5418 Å).

3. The crystalline form of tezacaftor according to claim 2, characterized by an XRPD profile comprising at least the peaks at 5.6, 11.3, 12.9 and 15.9 degrees 2θ, when collected with the Kα radiation of copper (λ=1.5418 Å).

4. The crystalline form according to claim 1, characterized by an XRPD profile additionally comprising at least one peak selected from the group consisting of 16.3, 19.7, 20.7, 21.3, 26.9 and 31.4 degrees 2θ, when collected with the Kα radiation of copper (λ=1.5418 Å).

5. The crystalline form according to claim 1, characterized by a differential scanning calorimetry thermogram at a heating rate of 10 ° C./min comprising a melting endotherm at 110-128° C.

6. (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane carboxamide (tezacaftor) in solid form according to claim 1, substantially free of any amorphous and other crystalline form.

7. A process for the preparation of crystalline tezacaftor according to claim 1 (form B), said process comprising the following steps:
a) providing a solution of tezacaftor in at least one organic solvent;
b) bringing into contact the solution of tezacaftor provided in step a) with an antisolvent so as to cause its precipitation in the crystalline form B; and
c) isolating and optionally drying the solid obtained in step b);
wherein the at least one organic solvent in step a) is selected from the group consisting of a C1-C8 linear or branched alkyl alcohol, a C4-C10 linear or cyclic aliphatic or aromatic ether, a C3-C10 linear or branched alkyl ketone, and mixtures thereof, and wherein the antisolvent in step b) is selected from the group consisting of a C5-C15 linear or branched alkane, a C6-C10 aromatic solvent, and mixtures thereof.

8. The process according to claim 7, wherein in step b) the solution of tezacaftor provided in step a) is added to the antisolvent so as to obtain precipitation of tezacaftor form B.

9. The process according to claim 8, in which the at least one organic solvent is at least one linear or cyclic aliphatic ether.

10. The process according to claim 9, in which the antisolvent used in step b) is an aliphatic hydrocarbon.

11. The process according to claim 10 in which the solution of tezacaftor is brought into contact with the antisolvent maintained at a temperature not higher than 15° C. and/or the volume ratio between the antisolvent in step b) and the at least one organic solvent in step a) is from 30:1 to 100:1.

12. A pharmaceutical formulation comprising the crystalline form of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropane-1-carboxamide (tezacaftor, Form B) according to claim 1 in mixture with at least one pharmaceutically acceptable excipient.

13. The pharmaceutical formulation according to claim 12 that is an oral dosage form selected from the group consisting of tablet, capsule, granules, powder, troche, syrup, emulsion, and suspension.

14. (canceled)

15. A method for preparing an alternative form of tezacaftor, the method comprising:
using the crystalline form B of tezacaftor according to claim 1 as a material for preparing the alternative form of tezacaftor,
wherein the alternative form is amorphous tezacaflor or a crystalline Form A of tezacaftor;
wherein the amorphous form is characterized by an XRPD profile having a halo pattern between 12° and 32° 2θ; and wherein the crystalline Form A is characterized by an XRPD profile having peaks at about 10.0°, 17.1°, 20.75°, 18.8°, 19.5°, 20.4°, 21.7° and 24.7° 2θ.

16.-30. (canceled)

31. The process according to claim 9, in which the at least one linear or cyclic aliphatic ether is selected from the group consisting of diethyl ether, tetrahydrofuran, 2-methyltetrahydrofuran, 3-methyltetrahydrofuran, and mixtures thereof.

32. The process according to claim 31, in which the at least one linear or cyclic aliphatic ether is 2-methyltetrahydrofuran.

33. The process according to claim 10, in which the aliphatic hydrocarbon is selected from the group consisting of pentane, hexane, cyclohexane, heptane, octane, and mixtures thereof.

34. The process according to claim 11, in which the volume ratio between the antisolvent in step b) and the at least one organic solvent in step a) is from 35:1 to 80:1.

35. The method according to claim 15, in which the alternative form of tezacaftor is Form A.

36. The method according to claim 15, in which the alternative form of tezacaftor is amorphous tezacaftor.
